(19) United States
(12) Patent Application Publication
(10) Pub. No.: US 2002/0052475 A1
(43) Pub. Date: May 2, 2002

(54) High affinity soluble interleukin-18 receptor

(75) Inventors: Stewart Leung, El Cerrito, CA (US); H. Daniel Perez, Kentfield, CA (US); Neil Miyamoto, Corte Madera, CA (US)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 09/908,494

(22) Filed: Jul. 19, 2001

Related U.S. Application Data
(60) Provisional application No. 60/219,447, filed on Jul. 20, 2000.

(51) Int. Cl. C07K 14/705; C07H 21/04; C12P 21/04; C12N 5/06

(52) U.S. Cl. 530/350; 536/23.5; 435/69.52; 435/320.1; 435/325

(57) ABSTRACT

A soluble, heterodimeric interleukin 18 (IL-18) receptor molecule is described which comprises two subunits, one of which comprises an extracelluar domain, or a fragment thereof, of IL-18R, and the other of which comprises an extracelluar domain, or a fragment thereof, of AcPL. Preferably, the soluble, heterodimeric receptor binds to IL-18 with higher affinity than does either IL-18R or AcPL alone.

This application claims the benefit of the filing date of U.S. Provisional application Ser. No. 60/219,447, filed Jul. 20, 2000.

### FIELD OF THE INVENTION

This invention relates, e.g., to soluble multimeric, including heterodimeric, interleukin 18 (IL-18) receptor molecules.

### BACKGROUND OF THE INVENTION

Interleukin-18 (IL-18), formerly called IFN-γ (interferon gamma)-inducing factor, is a cytokine which exhibits many biological activities. These biological activities are mediated by the binding of IL-18 molecules to cell surface, or plasma membrane, receptors on cells, e.g., activated T-or NK-cells. IL-18 receptors comprise at least two subunits: IL-18R (also known as IL-IR-related protein, IL-1Rrp, IL-18Rα, 2FI or the “binding chain”) and AcPL (also known as accessory protein-like, IL-18-AcPL, IL-18Rβ or the “signalling chain”). There is a need for agents which bind to IL-18 with high affinity. Such agents can be used, e.g., to detect IL-18 in a sample or to treat pathological conditions mediated by IL-18, e.g. by interfering with the binding of IL-18 to IL-18 receptors located on the surface of cells.

### DESCRIPTION OF THE INVENTION

This invention relates, e.g., to soluble, multimeric IL-18 receptor molecules (sIL-18R), comprising at least two subunits, one of which comprises an extracellular domain, or a fragment or variant thereof, of the receptor IL-18R, and another of which comprises an extracellular domain, or a fragment or variant thereof, of the receptor AcPL, wherein said soluble receptor molecules bind to IL-18 with a higher affinity than does either the IL-18R or the AcPL polypeptide, alone. Preferably, the sIL-18R exhibit high affinity binding to IL-18. In a preferred embodiment, the soluble IL-18 receptor is heterodimeric, containing two different subunits as described above.

By “soluble” multimeric receptor is meant herein a multimeric receptor, each of whose subunits comprises part or all of an extracellular domain of a receptor, but lacks part or all of the transmembrane domain which normally retains the fall length receptor in the cell membrane. Thus, for example, when such a soluble receptor or one of its subunits is produced recombinantly in a mammalian cell, it can be secreted from the recombinant host cell through the plasma membrane, rather than remaining at the surface of the cell. In general, a soluble receptor of the invention is soluble in an aqueous solution. However, under certain conditions, the receptor can be in the form of an inclusion body, which is readily solubilized by standard procedures. By “multimeric” or “heteromultimeric” is meant comprising two or more different subunits. A “heterodimeric” receptor contains two different subunits.

In a preferred embodiment of the invention, one subunit of a heterodimeric IL-18 receptor exhibits one or more of the biological functions (activities) of IL-18R, and the other exhibits one or more of the biological functions (activities) of AcPL. The term, a “biological activity” of IL-18R, as used herein, means the ability to bind to IL-18, at least to some extent, and/or to modulate (e.g., enhance, change) the binding of AcPL, or another molecule, to IL-18. The term, a “biological activity” of AcPL, as used herein, means the ability to bind, at least to some extent, to IL-18, and/or to modulate (e.g., enhance, change) the binding of IL-18R, or another molecule, to IL-18. Other polypeptides, or fragments or variants thereof, which bind to IL-18, and/or which modulate such binding, are also encompassed by the invention.

By “high affinity binding” is meant herein about 100 pM to about 1 nM. By “low affinity binding” is meant herein about 10 to about 100 nM. See Example 4 for a description of some assays for measuring IL-18 binding.

In one embodiment of the invention, IL-18R-and AcPL-containing subunits are associated by chemical cross-linking.

In another embodiment, IL-18R and AcPL subunits are associated via moieties, such as peptides, for example leucine zipers, portions of antibody molecules, or the like, which are appended to soluble portions of IL-18R and AcPL.

In another embodiment, IL-18R and the AcPL subunits form (are present in) a single polypeptide claim.

This invention also relates to a method for detecting IL-18 in a sample which may contain IL-18, comprising contacting the sample with a soluble, heterodimeric IL-18 receptor as above which is labeled, and detecting the label.

This invention also relates to a method of treating or preventing a condition (e.g., a pathological condition) associated with expression of IL-18, including excessive or inappropriate amounts thereof, and/or with excessive or inappropriate activity of cells possessing IL-18 receptors, comprising administering to a patient in need of such treatment an effective amount of a soluble, heterodimeric IL-18 receptor as above.

The soluble, multimeric IL-18 receptors of the invention can be prepared in any suitable manner, e.g., by preparing the subunits individually and then associating them, or by preparing a macromolecule which already comprises the subunits. Although it is to be understood that any receptor polypeptide which binds to IL-18 and/or which modulates the binding of a polypeptide to IL-18 is encompassed by the invention, for clarity this application disclosure is focused primarily on the receptors IL-18R and AcPL.

IL-18R and AcPL have been characterized, cloned and sequenced from both murine and human sources, and have been purified from many of them; and they have been at least characterized from other mammalian species such as, e.g., bovine, porcine and various non-human primate sources. For procedures to purify, manipulate and/or clone IL-18R and AcPL, and/or for a disclosure of their sequences, see, e.g., Dinarello (1999). J. Allergy Clin. Immunol. 103, 11-24; Torigoe et al. (1997) J. Biol. Chem. 272, 25,737-742; Parnet et al. (1996). J. Biol. Chem. 271 3967-70; EPs 864585 and 850952; WO97/31010; U.S. Pat. No. 5,776,731; or Greenfeder et al. (1995) J. Biol. Chem. 270,13,757-765; or Born et al (1998). J. Biol. Chem. 273, 29,445-450.

Functional domains of IL-18R and AcPL have been identified. For example, at least one isolate of human IL-18R comprises a signal peptide (amino acid −19 to −1), followed by an extracellular domain (amino acid 1-310), a transmembrane region (amino acid 311-332) and a cytoplasmic domain (amino acid 333-522) (WO 97/31010). At least one isolate of human AcPL comprises a signal peptide (amino acid −14 to −1), followed by an extracellular domain (amino acid 1-342), a transmembrane region (amino acid 343-367) and a cytoplasmic domain (amino acid 368-585) (Born et al. (1998) J. Biol. Chem. 273, 29,445-50). A subunit of a soluble, heterodimeric receptor of the invention can comprise, in addition to an extracellular domain or a fragment thereof of IL-18R or AcPL, sequences from the signal sequence, the transmembrane region, and/or the cytoplasmic domain of the receptor, or fragments of these regions, provided that biological activity as defined above is retained and the molecule remains soluble. These moieties need not be in the same linear arrangement or relative orientation as in the wild type molecule, and they can comprise internal deletions. A receptor subunit of the invention can contain all or part of a signal peptide sequence, or none at all. A molecule containing an extracellular domain of IL-18R or AcPL and,,optionally, one or more other moieties from the respective receptor polypeptide chains as defined above, wherein the molecule is soluble and exhibits activity as defined above, is designated herein as a “soluble portion of a receptor.”

To isolate soluble portions of receptor polypeptides, one can, using conventional, art-recognized procedures such a those disclosed in the above references, begin by isolating substantially full-length receptors. Substantially full-length IL-18R or AcPL can be isolated from a variety of in vivo sources (e.g., from lung, spleen, epithelial cells, endothelial cells, interstitial cells, chondrocytes, monocytes, granulocytes, lymphocytes, neurocytes, etc.), from established cell lines which express one or both of the proteins (e.g., hematopoietic cells, including lymphocytes, peripheral blood T cells and NK cells), from lymphoma cells which secrete one or both of the receptors or fragments thereof, or from recombinant cells which express and, optionally, secrete the polypeptides. By “substantially full-length” is meant containing all or nearly all of the receptor domains described above. Subsequently, one can isolate soluble portions of the substantially full-length proteins. For example, a soluble portion can be cleaved from a full length receptor polypeptide, whether or not it is bound to a cell, with one or more proteolytic enzymes (e.g., peptidases or proteases, such as trypsin, chymotrypsin, pepsin, bromelain, papain, bovine enterokinase, collagenase, factor IX, polyubiquitin processing enzyme, or the like) or by chemical cleavage (e.g., with cyanogen bromide, etc.). Naturally occurring soluble forms of the receptors (e.g., “decoy” receptors) can also be used to generate heterodimeric soluble receptors. Of course, one or more of the receptor domains can be prepared individually, as described above, and then joined to other domains, using art-recognized procedures, to form a “soluble portion of a receptor.”

Alternatively, soluble portions of either of the receptors can be prepared recombinantly, using conventional methods. As an initial step, polynucleotide fragments (e.g., DNA fragments) encoding such soluble portions are generated by any of a variety of procedures. For example, they can be cleaved from larger polynucleotides (e.g., genomic sequences, cDNA, or the like) with appropriate restriction enzymes, which can be selected on the basis of published sequences of human and murine IL-18R (see, e.g., Parnet et al, supra and U.S. Pat. No. 5,776,731) or human and murine AcPL (see, e.g., Born et al., supra). In another embodiment, polynucleotide fragments encoding such soluble portions can be generated by PCR amplification from longer templates, by selecting appropriate primers based on published sequences such as those above. Methods of PCR amplification, including the selection of primers, conditions for amplification, and cloning of the amplified fragments, are conventional. See, e.g., Innis, M. A. et al, eds. PCR Protocols: a guide to methods and applications, 1990, Academic Press, San Diego, Calif. and Wu et al., eds., Recombinant DNA Methodology, 1989, Academic Press, San Diego, Calif. In another embodiment, polynucleotide fragments encoding such soluble portions can be generated by chemical synthesis. Combinations of the above recombinant or non-recombinant methods, or other conventional methods, can also be employed. Of course, polynucleotide fragments corresponding to one or more of the receptor domains can be prepared individually as described above and then joined to fragments encoding other domains, using art-recognized procedures, to form a polynucleotide which corresponds to a “soluble portion of a receptor.”

Once a polynucleotide encoding a soluble portion of IL-18R or AcPL has been isolated, it can be cloned into any of a variety of expression vectors, under the control of a variety of regulatory elements, and expressed in a variety of cell types as hosts, including prokaryotes, yeast, and mammalian, insect or plant cells, or in a transgenic, non-human animal. In a preferred embodiment, the expressed soluble portions are secreted by the cell; either the natural or a heterologous leader sequence (signal peptide) can be employed to facilitate secretion.

Methods of cloning nucleic acids are routine and conventional in the art. For general references describing methods of molecular biology which are mentioned in this application, e.g., isolating, cloning, modifying, labeling, manipulating, sequencing and otherwise treating or analyzing nucleic acids and/or proteins, see, e.g., Sambrook, J. et al. (1989). Molecular Cloning, a Laboratory Manual. Cold Harbor Laboratory Press, Cold Spring Harbor, N.Y.; Ausubel, F. M. et al. (1995). Current Protocols in Molecular Biology, N.Y., John Wiley & Sons; Davis et al. (1986), Basic Methods in Molecular Biology, Elsevir Sciences Publishing,, Inc., New York; Hames et aL (1985), Nucleic Acid Hybridization, IL Press; Dracopoli, N.C. et al. Current Protocols in Human Genetics, John Wiley & Sons, Inc.; and Coligan, J. E., et al. Current Protocols in Protein Science, John Wiley & Sons, Inc. Other references which, in addition, disclose methods specifically drawn to cloning and characterizing receptor proteins include, e.g., U.S. Pat. Nos. 5,919,903, 5,536,657 and 5,776,731, EP 864 585, and WO 9731010.

Nucleic acid encoding soluble receptors or subunits thereof can also be cloned into plants or animals (e.g., murine species, rabbits, cows, pigs, goats, non-human primates or the like) to generate transgenic species; and the products expressed from the transgenes can be isolated. Methods to make and use transgenic organisms for this purpose are routine and are described, e.g., in Hogan et al., (1986) Manipulating The Mouse Embryo, Cold Spring Harbor Press; Krimpenfort et al., (1991) Bio/Technology 9, 86; Palmiter et al., (1985) Cell 41, 343; Kraemer et al., (1985) Genetic Manipulation of The Early Mammalian Embryo, Cold Spring Harbor Laboratory Press; Hammer et al., (1985) Nature 315, 680; Purcel et al., (1986) Science 244, 1281; Wagner et al., U.S. Pat. No. 5,175,385; and Krimpenfort et al, U.S. Pat. No. 5,175,384.

“IL-18R” and “AcPL” receptors and soluble portions thereof of the invention encompass a variety of variants (derivatives) or fragments of wild type human or murine receptors, either naturally occurring or deliberately generated, wherein the changes do not substantially alter the normal function of the polypeptides, as defined elsewhere herein.

Such variant polypeptides exhibit substantial identity to comparable portions of wild type murine or human receptors. The term “substantial identity” or “substantial similarity” as used herein indicates that a polypeptide (or a nucleic acid) comprises a sequence that has at least about 90% sequence identity to a reference sequence, or preferably at least about 95%, or more preferably at least about 98% sequence identity to the reference sequence, over a comparison window of at least about 10 to about 100 or more amino acids residues or nucleotides. An indication that two polypeptide sequences are substantially identical is that one protein is immunologically reactive with antibodies raised against the second protein. An indication that two nucleic acid sequences are substantially identical is that the polypeptide which the first nucleic acids encodes is immunologically cross reactive with the polypeptide encoded by the second nucleic acid.

Variant polypeptides of the invention include polypeptides having one or more naturally-occurring (e.g., through natural mutation) or non-naturally-occurring (e.g., by deliberate modification, such as by site-directed mutagenesis) modifications, e.g., insertions, deletions and/or substitutions, either conservative or non-conservative. By “conservative substitutions” is meant by combinations such as Gly, Ala; Val, Ile, Leu; Asp, Glu; Asn, Gln; Ser, Thr; Lys, Arg; and Phe, Tyr. Variants can include, e.g., homologs, muteins and mimetics. Many types of protein modifications, including post-translational modifications, are included. Posttranslational modifications include naturally occurring or synthetically produced, covalent or aggregative conjugates with other chemical moieties, e.g., glycosyl groups, lipids, phosphates, acetyl groups, etc., as well as cleavage, such as of terminal amino acid(s). See, e.g., modifications disclosed in U.S. Pat. No. 5,935,835. The invention also encompasses variants such as polypeptides in which cysteine residues which are nonessential for biological activity are deleted or replaced with other amino acids, thereby preventing the formation of incorrect intramolecular disulfide bridges; naturally occurring variants arising from alternative mRNA splicing events; and altered forms reflecting genetic polymorphism (e.g., allelic variation). “Fragments” of the receptor polypeptides can be of any length, and from any portion of the polypeptide, provided that the truncated molecules retain the desired biological activity.

Soluble heterodimeric receptors of the invention, or subunits thereof, can comprise moieties coupled to or incorporated into at least one of the polypeptides, which can be useful for therapeutic purposes or for detection. Detectable moieties can be, e.g., radioisotopes, radionuclides, phosphorescent and fluorescent entities, bioluminescent markers, or the like.

The invention also relates to a nucleic acid corresponding to a subunit of a soluble, heterodimeric receptor of the invention (e.g., a fusion protein) or to a single chain soluble heterodimeric receptor. Such a nucleic acid can comprise both coding sequences and regulatory sequences which govern their expression. The nucleic acid sequence corresponding to a moiety of such a heterodimeric receptor (e.g., a soluble portion of a murine or human IL-18R or AcPL receptor, or a peptide linker) exhibits substantial identity to the nucleic acid encoding the corresponding wild type molecule. Properties which confer “substantial identity” upon two nucleic acids are defined above. A further indication that two nucleic acids exhibit substantial identity is that the two molecules hybridize to each other under selected high stringent conditions. High stringent conditions are sequence-dependent and will be different with different environmental parameters. Generally, high stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, high stringent conditions will be those in which the salt concentration is at least about 0.2 molar at pH 7 and the temperature is at least about 60° C.

A nucleic acid of the invention can include one or more naturally or non-naturally-occurring modifications, mutations, polymorphisms, etc.; and the nucleic acid can differ from its wild type counterpart with regard to base composition, reflecting the degeneracy of the genetic code.

A variety of conventional, art-recognized methods can be used to associate (e.g., bind, covalently or non-covalently; couple; attach; cross-link; join; connect) two (or more) different subunits to form a soluble heterodimeric (or multimeric) IL-18 receptor molecule. Typical methods of generating, purifying and characterizing soluble heterodimeric or multimeric receptors are disclosed, e.g., in W097/3 1010; WO99/37772; U.S. Pat Nos. 5,919,903; 5,470,952; Croze et al. (1996) Eur. Cytokine Network, First Joint Meeting of the ICS and ISICR; and Arduini et al. (1999) Protein Science 8, 1867-77. The general categories of methods which can be used to associate subunits to form the soluble heterodimers of the invention include, e.g., 1) coupling the subunits by chemical cross-linking; 2) appending a moiety, such as a peptide, to the soluble portions of IL-18R and AcPL to form fusion or hybrid proteins, and then joining the fusion or hybrid proteins via the appended moieties; and 3) linking soluble portions of IL-18R and AcPL to form a single polypeptide chain.

In the first category, any of a variety of conventional methods can be used to chemically couple (cross-link) the two polypeptide chains. Covalent binding can be achieved either by direct condensation of existing side chains (e.g., the formation of disulfide bonds between cysteine residues) or by the incorporation of external bridging molecules. Many bivalent or polyvalent agents are useful in coupling polypeptides.

In general, the cross-linking agents used are bifunctional agents reactive, e.g., with E-amino group or thiol groups. These cross-linkers can be classified into two categories: homo-and hetero-bifunctional reagents. Homobifunctional reagents can react, e.g., with free thiols (e.g., generated upon reduction of disulfide bonds), and include, e.g., 5,5′-Dithiobis (2-nitrobenzoic acid) (DNTB), and o-phenylenedimaleimide (O-PDM), which can form a thioether bond between two polypeptides having such free thiols. Heterobifunctional reagents can introduce a reactive group onto a polypeptide that will enable it to react with a second polypeptide. For example, N-succinimidyl-3-(2-pyridyldithio) propionate (SPDP) can react with a primary amino group to introduce a free thiol group. Other chemical cross-linking agents include, e.g., carbodiimides, diisocyanates, diazobenzenes, hexamethylene diamines, dimaleimide, glutaraldehyde, 4-succinimidyl-oxycarbonyl-α-methyl α(2-pyridylthio)toluene (SMPT) and N-succinimidyl-S acetyl-thioacetate (SATA). Procedures for cross-linking polypeptides with such agents are well-known in the art. See, e.g., Pierce ImmunoTechnology Catalog & Handbook (1991) E8-E39; Karpovsky et al. (1984) J. Exp. Med. 160, 1686; Liu et al. (1985) Proc. Natl. Acad. Sci. 82, 8648; Segal et al. U.S. Pat. No. 4,676,980 (Jun. 30, 1987) and Brennan (1986) Biotech. 4, 424.

Spacer arms between the two reactive groups of cross-linkers may have various lengths and chemical compositions. A longer spacer arm allows a better flexibility of the conjugated polypeptides. while some particular components in the bridge (e.g., a benzene group) may lend extra stability to the reactive groups or an increased resistance of the chemical link to the action of various aspects (e.g., disulfide bond resistance to reducing reagents). The use of peptide spacers such as the peptide linkers or linker peptides described below is also contemplated.

In the second category of association methods, conventional methods can be used to append any of a variety of moieties (e.g., peptides, sometimes called herein “peptide linkers” or “fusion domains”) to soluble portions of the receptors of interest, thereby generating hybrid or fusion proteins, and the hybrid or fusion proteins can then be associated via the appended moieties. Some of the many types of possible associations via appended peptides are illustrated in FIG. 1B.

In one embodiment, moieties such as biotin and avidin (streptavidin) are complexed to soluble portions of the receptors, using conventional methods, and these moieties interact to associate the two subunits.

In a preferred embodiment, the appended moieties are peptides (“peptide linkers”). Among the wide variety of peptide linkers which can be used are the GST (glutathione S-transferase) fusion protein, or a dimerization motif thereof; a PDZ dimerization domain; FK-506 BP (binding protein) or a dimerization motif thereof; a natural or artificial helix-turn-helix dimerization domain of p53; and Protein A or its dimerization domain, domain B.

In a most preferred embodiment, the appended peptides are components of a leucine zipper. The leucine zipper moieties are often taken from the human transcription factors c-jun and c-fos. Example 5 demonstrates the use of a leucine zipper to generate a heterodimeric sIL-18R.

In another most preferred embodiment, the appended peptides are portions of immunoglobulins, e.g., IgA, IgM, IgD, IgE, or, preferably, IgG. For example, to one soluble receptor portion is appended a peptide containing a constant region, or fragment thereof, of a heavy chain, and to another is appended a corresponding constant region, or fragment thereof, of a light chain, preferably wherein thiol-containing amino acids from the hinge region are present. In this way, the two appended moieties can bind to one another, e.g., via disulfide bonds, to generate a molecule resembling an Fab fragment with soluble receptor moieties attached to each of the antibody chains. In another embodiment, the appended moieties are portions of immunoglobulin heavy chains (e.g., comprising the CH₂ moiety and/or the CH₃ moiety of the constant region), which can associate via the disulfide bonds that are normally responsible for dimerizing heavy chains, thereby forming a molecule resembling an Fc fragment with a soluble receptor portion attached to each of the antibody chains. Example 6 demonstrates the use of receptor subunits comprising appended IgG fragments to generate a heterodimeric sIL 18R.

Of course, two subunits can be associated via any combination of the above moieties, e.g., a tandem arrangement of a leucine zipper moiety and an immunoglobulin domain, arranged in any relative order or orientation.

“Peptide linkers” of the invention encompass any of the types of fragments or “variants” described above with regard to “IL-18R” and “AcPL” receptors.

A peptide linker should provide an adequate degree of flexibility to prevent the two subunits from interfering with each others' activity, for example by steric hindrance, and to allow for proper protein folding; yet it should allow the two subunits to interact with one another as necessary in order to enable the proper spatial orientation for binding to IL-18, preferably at a high affinity. Therefore, it may be desirable to modify a peptide linker by altering its length, amino acid composition, and/or conformation, e.g., by appending to it still other “secondary linker moieties” or “hinge moieties.” Among the many types of secondary linker moieties are, e.g., tracts of small, preferably neutral and either polar or nonpolar, amino acids such as, e.g., glycine, serine, threonine or alanine, at various lengths and combinations; polylysine; or the like. Alternatively, multiples of linkers and/or secondary linker moieties can be used. It is sometimes desirable to use a flexible hinge region, such as, e.g., the hinge region of human IgG, or polyglycine repeats interrupted by serine or threonine at certain intervals.

The length and composition of a peptide linker can readily be selected by one of skill in the art in order to optimize the desired properties of the soluble receptor, e.g., its ability to bind to IL-18. Conventional assays for binding to IL-18 are described, e.g., in Example 4 and in Thomassen et aL (1998). J. Interferon Cytokine Res. 18, 1077-1088 (IL-18 binding to heterodimer linked to BlAcore chip); Torigoe et al. (1997). J. Biol. Chem. 272, 25737-25742 (I¹²⁵-IL-18 binding to cells); Born et al. (2000). J. Immunol. 164, 3246-3254 (I¹²⁵-IL-18 binding to heterodimer followed by immobilization on protein A); Born et al. (1998). J. Biol. Chem. 273, 29445-29450 (inhibition of IL-18 induced NF-κB reporter gene activation); Novick et al. (1999). Immunity 10, 127-136 (inhibition of IL-18 induced IFN-γ production (which can also be measured in animals)); and WO97/31010 (an indirect assay, measuring the inhibition of prostaglandin E2 synthesis, either in vitro or in animals).

Peptide linkers can be appended to soluble portions of receptors to form hybrid or fusion molecules by a variety of methods which will be evident to one of ordinary skill in the art, e.g., chemical coupling as described above (if necessary, following derivatization of appropriate amino acid groups); attachment via biotin/avidin interactions; covalent joining of the peptides by art-recognized methods (e.g., using appropriate enzymes); recombinant methods; or combinations thereof. “Hybrid” proteins of the invention are proteins in which a moiety comprising a soluble portion of a receptor and moiety comprising a linker peptide are joined via linkages other than peptide linkages (e.g., by chemical coupling or via biotin/avidin interactions). “Fusion” proteins of the invention are proteins in which such moieties are linked by peptide bonds, preferably accomplished by recombinant processes.

Methods of making recombinant fusion proteins are conventional and are described, e.g., in Ashkenazi et al (1991) PNAS 88, 10535; Byrn et al. (1990) Nature 344, 677; Hollenbaugh et al. (1992) “Construction of Immunoglobulin Fusion Proteins,” in Current Protocols in Immunology, Suppl. 4, pp. 10.19.1 to 10. 19.1 1; WO93/1015 1; and U.S. Pat. No. 5,457,035. Typical methods are shown in Examples 5 and 6. Each of the fusion proteins can be expressed independently in a single expression vector, or two or more fusion proteins can be expressed in the same expression vector. Typically, sequences encoding the two polypeptide moieties of a fusion protein are fused in frame. Generally, fusion proteins are marked with selectable markers, in order to facilitate the selection of transfectants (transformants).

The soluble receptor portions can be oriented in each subunit so that, when the two subunits are associated, the soluble receptor portions are joined via either their N-termini or their C-termini, provided that the linkage does not interfere with the ability of one or both of the subunits to exhibit a desired biological activity. In a preferred embodiment, the two soluble receptor portions are joined via their C termini, in order to minimize physical constraints on the “working portions” of the molecules, which lie within their N-terminal regions. See FIG. 1B for illustrations of some of the possible types of orientations.

Pairs of hybrid or fusion molecules formed as described above can be associated with each other via the appended moieties by non-covalent or covalent bonds. The non-covalent bonds include, e.g., leucine zippers, biotin/avidin interactions, hydrogen bonding, van der Waals forces, hydrophobic interactions, etc. Among possible covalent bonds are, e.g., naturally forming disulfide bonds (e.g., formation of modified Fab or F(ab′)2 fragments), or bonds formed by chemical cross-linking reactions as described above. The attachment can occur in vitro (e.g., in a test tube) or within a cell.

In a preferred embodiment, the attachment occurs intracellularly. Two separate chimeric polynucleotides, each encoding one of two different fusion proteins, are transfected into and co-expressed in the same host cell. Fusion polypeptides so produced are believed to join to one another within the cell or during secretion. They are then purified from a cell lysate or, preferably, are secreted from the cell and are purified from the culture medium. See, e.g., Examples 5 and 6. The two fusion proteins can be expressed either from the same expression vector or from two different expression vectors.

If desired, the relative amounts of two recombinant fusion proteins produced in a cell can be regulated, e.g., by expressing them from promoters of different strengths. For example, if the appended peptide of subunit A forms homodimers at a high frequency, whereas the appended peptide of subunit B forms homodimers at a low frequency, one can drive the formation of the desired heterodimers by expressing much higher levels of subunit B than of A. The optimal relative amounts can be determined empirically by routine experimentation.

The invention also relates to a chimeric polynucleotide encoding a fusion protein as described above, a host cell expressing such a fusion protein, and a method of making such a fusion protein comprising culturing such a cell under conditions in which the fusion protein is expressed and harvesting (recovering) the protein. A fusion protein of the invention can also be made by in vitro translation of a chimeric polynucleotide as above. The invention also relates to antibodies (e.g., monoclonal antibodies) immunorective with novel hybrid or fusion proteins of the invention.

In the third category of association methods, recombinant techniques are used to join soluble portions of each of two receptors, in frame, to form a single chain polypeptide molecule. FIG. 1C illustrates some of the possible combinations. Preferably, the receptor portions are separated from one another by a linker peptide, of any length or amino acid composition, most preferably a flexible loop structure, which allows the two receptor moieties to lie at an appropriate distance from each other and in a proper alignment for optimal interaction. Typical linker peptides contain tracts of small, preferably neutral and either polar or nonpolar amino acids such as, e.g., glycine, serine, threonine or alanine, at various lengths and combinations; polylysine; or the like. The linker peptide can have at least one amino acid and may have 500 or more amino acids. Preferably, the linker is less than about 100 amino acids, most preferably about 10 to 30 amino acids. Flexible linker domains, such as the hinge region of human IgG, or polyglycine repeats interrupted by serine or threonine at certain intervals, can be used, alone or in combination with other moieties.

Recombinant methods which can be used to generate such linear, single chain heterodimeric receptors are conventional. Furthermore, routine procedures can be used to select linker peptides and to optimize parameters so that the two soluble receptor portions are aligned at a distance and in an orientation which allow optimal function of the soluble, heterodimeric receptor. See, e.g., U.S. Pat. No. 4,935,233 and 4,751,180.

The invention also relates to a chimeric polynucleotide which encodes a single chain heterodimeric soluble receptor molecule as described above; a host cell expressing such a protein; a method of making such a protein, comprising culturing such a cell under conditions in which the protein is expressed and harvesting (recovering) the protein; and an antibody (e.g., a monoclonal antibody) immunoreactive with such a novel single chain polypeptide. A single chain heterodimeric soluble receptor of the invention can also be made by in vitro translation of such a chimeric polynucleotide.

In addition to the heterodimeric receptors described above, soluble multimeric IL-18 receptors can be made by extrapolating any of the above methods, or combinations thereof, to join three or more receptor subunits, in any combination (e.g., 2 copies of IL-18R and one copy of AcPL; two copies each of IL-18R and AcPL; 3 or 4 copies of each of IL-18R and AcPL, etc.). Some of the possible variations are summarized in FIG. 1D.

Preferably, a soluble dimeric receptor of the invention is “isolated,” e.g., is in a form other than it occurs in nature, for example in a buffer, in a dry form awaiting reconstitution, as part of a kit or a pharmaceutical compostion, etc.

A variety of conventional methods can be used to isolate and/or purify a soluble receptor of the invention. The desired degree of purity may depend on the intended use of the protein. For example, in vitro IL-18 binding studies are sometimes performed with the supernatants from cells transfected with a vector encoding one or more of the receptor subunits. Typically, the receptor is substantially purified. The term “substantially purified,” as used herein, refers to a receptor which is substantially free of contaminating endogenous materials, such as, e.g., other proteins, lipids, carbohydrates, nucleic acids and other biological materials with which it is naturally associated. For example, a substantially pure molecule can be at least about 60%, by dry weight, preferably about 70%, 80%, 90%, 95% or 99% the molecule of interest.

Soluble receptors of the invention and/or their subunits can be recovered from cells either as soluble proteins (preferably after having been secreted into the culture fluid) or as inclusion bodies, from which they may be extracted quantitatively, e.g., by 8M guanidium hydrochloride and dialysis. Conventional purification methods which can be used include, e.g., ion exchange chromatography, hydrophobic interaction chromatography, reverse phase chromatography, and/or gel filtration. In a preferred embodiment, affinity chromatography is used, e.g., with a column containing IL-18 or another appropriate ligand; an appropriate lectin, such as, e.g., wheat germ agglutinin; protein A or protein G, which can bind to the Fc moieties present in certain soluble receptors; or antibodies specific for IL-18R and/or AcPL. In a particularly preferred embodiment, each receptor is “tagged” with a moiety, preferably a cleavable one, that can bind to an appropriate affinity column. For example, one or both subunits can be tagged with poly His (e.g., His₆) to allow rapid purification by metal-chelate chromatography; with a Strep-tag which binds to streptavidin and can be eluted with iminobiotin; with maltose binding protein (MBP), which binds to amylose and can be eluted with maltose; or with any other such moiety which can be separated by affinity chromatography. Alternatively, one can tag one or both of the subunits with epitopes to which antibodies are available, such as the FLAGS® peptide, Asp-Tyr-Lys-Asp-Asp-Asp-Asp-Lys (available from Eastman Kodak Co., Scientific Imaging Systems Division, New Haven, Conn.). Other such antigenic identifiers are described in U.S. Pat. No. 5,011,912 and in Hopp et al. (1988) Bio/Technology 6, 1204. (The attachment of such epitopes can also allow for, e.g., selective immunoprecipitation, detection on western blots, or activity depletion/blocking in bioassays.) For typical methods of using affinity tags, see, e.g., Recombinant Protein Protocols: Detection and Isolation, Edited by Rocky S. Tuan, Methods in Molecular Biology, Vol. 63, Humana Press, 1997 and Examples 5 and 6. Combinations of any of the above types of tags can be used, of course.

If the method of preparation of a dimeric receptor results in the formation of homodimers as well as heterodimers, the desired heterodimers can be separated from the homodimers by any of a variety of procedures which allow differentiation between the two forms, e.g., chromatographic techniques or passive elution from preparative, non-denaturing acrylamide gels. In a most preferred embodiment, each of the subunits is tagged with a different tag, and doubly tagged dimeric soluble receptors are separated from singly tagged homodimers by affinity chromatography. See, e.g., Examples 5 and 6.

The purity of the receptors can be determined using standard methods including, e.g., polyacrylamide gel electrophoresis, column chromatography, and amino-terminal amino acid sequence analysis.

This invention also relates to a method of treating or preventing a condition (e.g., a pathological condition) associated with expression of IL-18, including excessive or inappropriate amounts thereof, and/or with excessive or inappropriate activity of cells possessing IL-18 receptors, comprising administering to a patient in need of such treatment an effective amount of a soluble, heterodimeric IL-18 receptor as above. Although not wishing to be bound to any mechanism, it is suggested that the soluble, heterodimeric IL-18 receptors of the invention may act as IL-18 antagonists, by binding IL-18, thus functionally inactivating it, and/or by competing for the binding of endogenous IL-18 to its natural receptor. Alternatively, again not wishing to be bound to any mechanism, it is suggested that the soluble, heterodimeric IL-18 receptors of the invention may act as IL-18 agonists, e.g., by acting as a “sink” to sequester IL-18 at particular sites in the body, thereby increasing the effective concentration of IL-18 at those sites.

Activities of IL-18 include, e.g., induction of natural killer (NK) cell cytotoxicity; enhancement of cytolytic T-cell responses; stimulation of the proliferation of activated T and NK cells; regulation (stimulation or repression of) a number of cytokines, including induction of IFN-γ by resting and activated T- and NK-cells; promotion of Tₕ-1 -type helper cell responses; and inhibition of osteoclast proliferation. Blocking or modifying IL-18 by contacting it with a soluble IL-18 receptor of the invention can modulate any of these, or other, activities mediated by IL-18, and thus can be used to ameliorate conditions or disorders mediated, directly or indirectly, by IL-18. A disorder is said to be mediated by IL-18 when IL-18 causes (directly or indirectly) or exacerbates the disorder.

Among the many IL-18 related conditions which can be treated or prevented by administering to a patient in need thereof a soluble dimeric receptor of the invention are a variety of inflammatory conditions (e.g., chronic inflammation), immune disorders (e.g., autoimmune or alloantigen-induced) and allergic diseases. Among the conditions which can be treated or prevented are, e.g, hepatotoxicity associated with endotoxemia, septic shock, autoimmune demyelinating diseases, including multiple sclerosis, rheumatoid arthritis, Crohn's disease, lupus nephritis, psoriasis, asthma, pernicious anemia, atrophic gastritis, Wegener granulomatosis, discoid lupus erythematosus, ulcerative colitis, inflammatory bowel disease, hyperthyroidism, autoimmune hemolytic anemia, myasthenia gravis, systemic lupus erythematosus, Addison's disease, Hodgkin's disease, various leukemias (including, e.g., ALL, CLL, AML and CML), HIV infections, septic shock which results from production or administration of excessive IFN-γ, insulin-resistant and juvenile onset diabetes, atopic dermatitis, and acute or chronic transplant rejection (e.g., Graft-versus-Host disease).

One of skill in the art can measure activity of the soluble dimeric receptors of the invention in any of a variety of suitable in vitro or cell culture assays, or in animal models. For example, one can determine whether such a receptor acts as an antagonist or an agonist, and can quantitate the amount of activity. Several such assays are discussed herein. Other in vivo methods include, e.g., systems for evaluating graft vs. host reactions (see, e.g., Fanslow et al. (1990) Science 248, 739-741 and animal models (e.g., the EAE model)for autoimmune demyelinating diseases such as, e.g., multiple sclerosis. For a description of animal models of MS, see, e.g., Gold et al. (2000). Mol Med. Today 6, 88-91 and Swanborg (1995). Clin. Immunol. Immunopathol. 21, 4-13. For a description of some methods of using the EAE animal model to test soluble receptors, see, e.g., Jacobs et al (1991). J. Immunol. 146, 2983-2989 and Selmaj et al (1995). J. Neuroimmunol. 56, 135-141.) See also Dinarello (1999) J. Allergy Clin. Immunol. 103, 11-24.

Soluble dimeric IL-18 receptors of the invention can be administered using conventional doses and delivery methods, such as those described for other, comparable therapeutic agents.

Dosages to be administered can be determined by conventional procedures known to those of skill in the art. See, e.g., The Pharmacological Basis of Therapeutics, Goodman and Gilman, eds., Macmillan Publishing Co., New York. In general, effective dosages are those which are large enough to produce the desired effect, e.g., blocking the binding of endogenous IL-18 to the natural receptor. The dosage should not be so large as to cause adverse side effects, such as unwanted cross-reactions, anaphylactic reactions, and the like. Factors to be considered include the activity of the specific agent involved, the metabolic stability and length of action of the agent, mode and time of administration, drug combination, rate of excretion, the species being treated, and the age, body weight, general health, sex, diet, and severity of the particular disease-states of the host undergoing therapy. For example, appropriate therapeutic regimens for a receptor of the invention involve administration to a patient of a dose of between about 1 ng/kg/day and about 10 mg/kg/day.

Appropriate methods of administration include parenteral and non-parenteral routes of administration. Parenteral routes include, e.g., intravenous, intraarterial, intraportal, intramuscular, subcutaneous, intraperitoneal; intraspinal, intrathecal, intracerebroventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection.

Ingredients, including excipients, diluents and/or carriers, for pharmaceutical compositions useful for the various modes of administration are conventional in the art, and are described, e.g., in Remington's Pharmaceutical Sciences, 18 th ed., Mack Publishing Company, 1990. The receptors can be formulated, e.g., in a pharmacologially acceptable liquid, solid or semi-solid carrier, linked to a carrier or targeting molecule (e.g., antibody, hormone, growth factor, etc.) and/or incorporated into liposomes, microcapsules or controlled release preparations (including cells which express the heterodimeric receptors) prior to administration in vivo.

The invention also relates to methods of detecting IL-18 molecules (e.g., experimental or diagnostic methods), comprising contacting a sample which may contain IL-18 molecules with a soluble, heterodimeric IL-18 receptor molecule of claim 1, which is labeled. Conventional moieties can be use to label the receptors, e.g., radioactive or fluorescent entities and to detect the labels. Such assays can be quantitative, of course. In one embodiment, such assays are used to determine whether an agent of interest causes an increase or decrease in a cell of the amount of IL-18 which is available for binding to a receptor (e.g., human or murine cells; in a test tube, in culture, or in an animal), and/or whether it modulates (inhibits or enhances) the biological activity of IL-18 (e.g., its binding to a soluble receptor). In some embodiments, cross-species reagents can be used, e.g., mouse receptors which bind to human IL-18.

Assays of the invention can be used, e.g., for experimental characterization of an agent; for screening for potentially therapeutic agents; for the diagnosis of diseases which can be indicated by the levels of IL-18 in bodily fluids; or to monitor the effects of treatment.

### BRIEF DESCRIPTION OF THE DRAWINGS

Various other features and attendant advantages of the present invention will be more fully appreciated as the same becomes better understood when considered in conjunction with the accompanying drawings:

FIG. 1 shows some examples of heterodimers and multimers. Panel A illustrates chemical cross-linking; Panel B illustrates linkage via appended moieties (generic appended peptides; heavy/light chain interactions of antibody fragments; heavy/heavy chain interactions of antibody fragments); Panel C illustrates single chain polypeptides; and Panel D illustrates tetramers.

FIG. 2A shows IL-18 responsiveness in 293 cells transfected with IL-18R and/or AcPL expression vectors.

FIG. 2B shows IL-18 responsiveness of cells which express IL-18R and which are transfected with an AcPL expression vector.

FIG. 3 shows that IL-12 and IL-18 synergistically induce IFN-γ secretion in Ae7 cells.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosure of all applications, patents and publications, cited above or below and in the figures are hereby incorporated by reference.

### EXAMPLES

1. Cloning of the human and mouse IL-18 receptors

The human and mouse IL-18 receptors can be cloned by reverse transcription-polymerase chain reaction (RT-PCR). For example, the mouse receptor subunits IL-18R (Accession #U43673) and AcPL (Accession #AF077347) are cloned as follows: total RNA is prepared from the mouse T cell clone Dorris, which is derived from AKR/J mice. RT is performed using the Clontech “Advantage RT-for-PCR kit”using an oligo-dT primer, and subsequent PCR is performed using primers corresponding to the 5′ and 3′ ends of the coding sequences of the two subunits. The full-length cDNA are cloned, via restriction enzymes sites enginereered into the 5′ and 3′ primers, into the eukaryotic expression vectors pcDNA3.1 (−)MYCHISB or pcDNA3.1(−)PUR (Invitrogen). The sequences of the mouse IL-18R and AcPL cDNAs are confirmed.

2. Generation of stable cell lines expressing mIL-18R

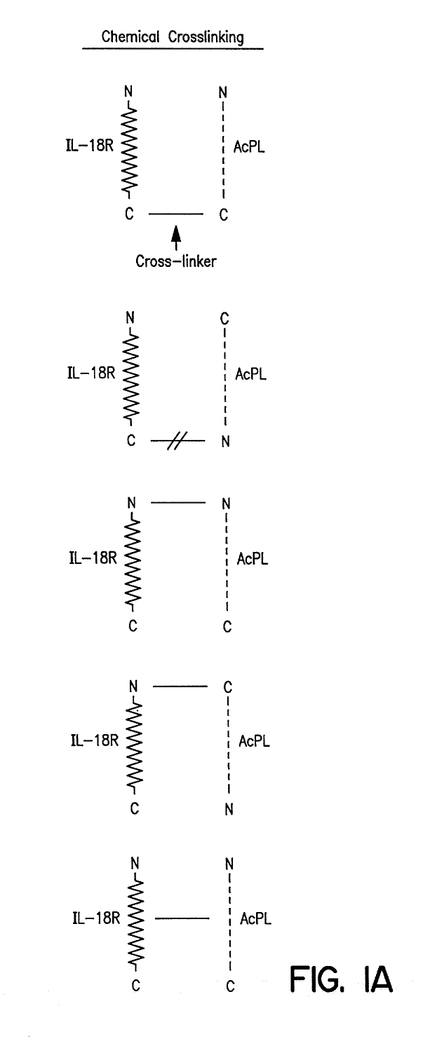

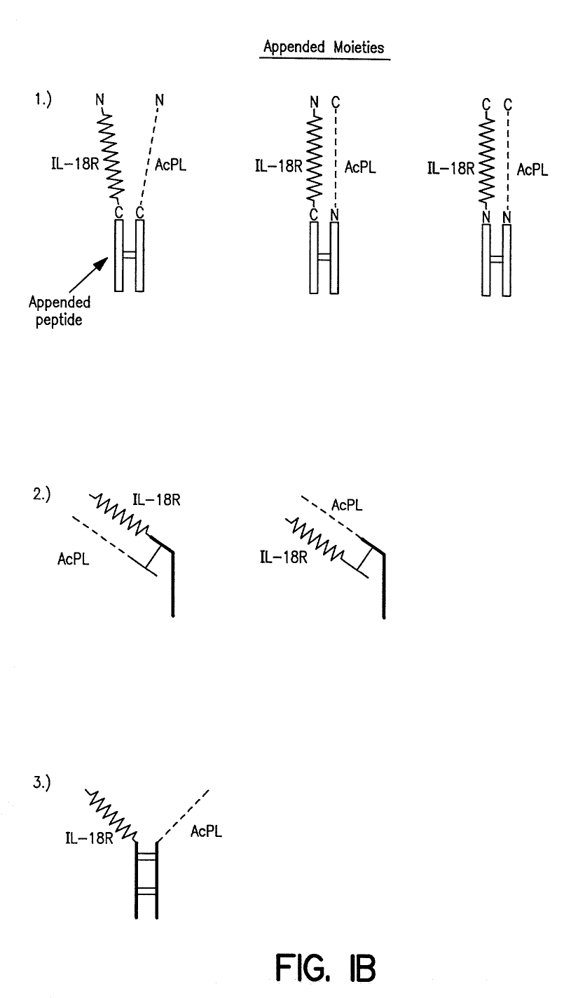

The human embryonic kidney fibroblast line 293 does not respond to mouse IL-18. FIG. 1A shows that responsiveness to mouse IL-18 (Peprotech) in 293 cells is optimal following the transient transfection of both mouse IL-18R and AcPL expression vectors. 293 cells are co-transfected with an NF-κB dependent luciferase reporter gene plasmid (Clontech) together with IL-18R and AcPL expression vectors, as indicated, using Effectene (Qiagen). Twenty-four hours after transfection, cells are exposed to IL-18 for a further 4 hours. IL-18-induced NF-κB is measured by luciferase activity in cell lysates.

A stable, neomycin-resistant 293 clone is established that expresses full-length mouse IL-18R, as determined by Western blotting using an α-Myc antibody, which recognizes the C-terminal Myc tag on the recombinant protein. FIG. 1B shows that IL-18 responsiveness in this clone requires the transient transfection of mouse AcPL. Stable neomycin- and puromycin-resistant 293 clones that express both mouse IL-18R and AcPL are generated.

3. IL-18 binding studies

Stable clones of 293 cells expressing both IL-18R and AcPL are cultured in 6-well plates until confluent. Cells are washed IX with PBS. Cells are incubated with 100,000 cpm (˜10 nM) of I¹²⁵-IL-18 (Bolton-Hunter Reagent) for 15 min at RT in 0.5 ml PBS, and total binding is determined. Non-specific binding is determined by including 1 μM of cold IL-18 in the incubation. Cell extracts are prepared by lysis in 10% SDS. Free IL-18 is separated from bound IL-18 by spinning through mineral oil. The amount of bound IL-18 is determined by direct counting in a scintillation counter.

Significant specific binding can be observed, indicating that the high affinity IL-18 receptor comprises IL-18R and AcPL subunits.

| Binding (CPM ± SD) | Experiment #1 | Experiment #2 |
|---|---|---|
| Total | 15349 ± 142 | 13888 ± 582 |
| Non-specific | 8546 ± 162 | 4063 ± 380 |
| Specific | 6803 ± 304 | 9825 ± 962 |

4. In vitro assays to measure IL-18 binding and/or activity

(A) NF-κB formation. In the mouse thymoma line EL4, IL-18 binds to its cognate receptor resulting in rapid formation of the transcription factor NF-κB. NF-κB can be detected in nuclear extracts by an electromobility shift assay (EMSA). The inhibition of IL-18 binding can be measured by incubation of the protein with an α-IL-18 antibody or soluble IL-18 receptors prior to cell stimulation.

For example, mouse IL-18 is pre-mixed with either PBS, rabbit α-mouse IL-18 polyclonal antibody from Torrey Pine Inc., control rabbit serum IgG; rat α-mouse IL-18 monoclonal antibody from Research Diagnostic Inc., control rat IgG2a, or homodimeric IL-18R-Fc fusion protein. After incubation at 37° C. for 2 hours, the mixes are used to stimulate EL-4 cells, the final concentration of IL-18 is 50 ng/ml. After 30 minutes, cells are harvested, and nuclear extracts are prepared. EMSA is performed using an NF-κB consensus probe (Santa Cruz).

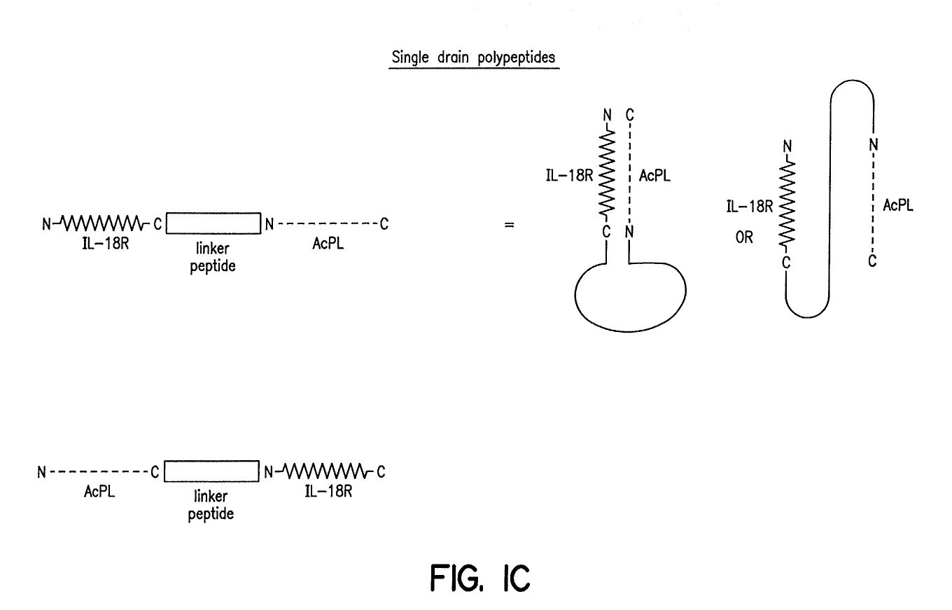

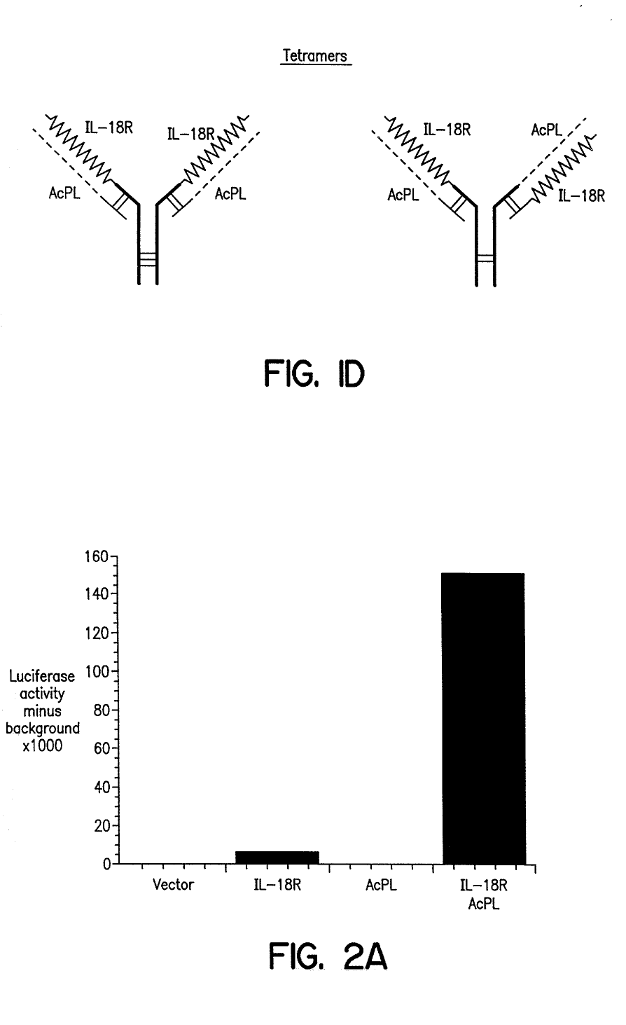

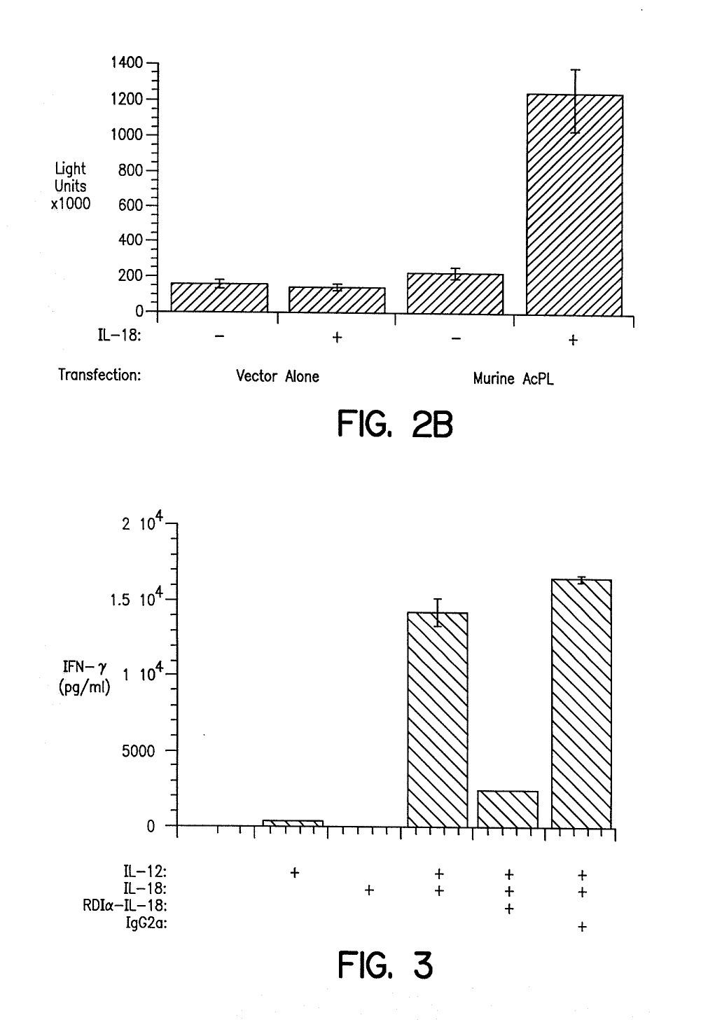

(B) NF-κB-dependent luciferase activity. FIGS. 2A and 2B show that IL-18 induces luciferase activity in 293 cells either transiently or stably expressing both mouse IL-18R and AcPL. 293 cells are transfected with a vector containing 3 copies of a consensus NF-κB binding site driving expression of the luciferase reporter gene.

Mouse IL-18 induces formation of NF-κB, which in turn activates the promoter driving the luciferase gene. The inhibition of luciferase activity is a functional demonstration of a soluble IL-18 receptor.

(C) IFN-γ release assay. The murine Th1 clone Ae7 synergistically responds to mouse IL-12 and IL-18 to secrete significant amounts of IFN-γ, measurable by standard ELISA (Biosource), as shown in FIG. 3. Pre-incubation of IL-18 with an equimolar amount of α-IL-18, but not control IgG, inhibits IFN-γ secretion. The assay can be used to test the ability of soluble IL-18 receptors to inhibit IFN-γ production.

In the Example shown in FIG. 3, Ae7 cells are primed with antigen and rested for 7 days, and then placed at 5×10⁵ cells/ml in 96-well plates. IL-12 (5 pM) or IL-18 (100 pM) are added. Rat α-mouse IL-18 monoclonal antibody or an isotype-matched rat IgG2a are preincubated with IL-18 (equimolar amounts) for 2 hours at 37° C. prior to adding to the cultures. After 48 hours, the amount of IFN-γ in the culture medium is measured using a Biosource IFN-γ Cytoscreen ELISA kit.

(D) Immunoprecipitation. The direct binding of α-IL-18 or soluble IL-18 receptors to IL-18 can be determined by co-immunoprecipitation experiments. For example, using a ax-Fc antibody, IL-18 can be co-precipitated with either α-IL-18 or the Fc-fusion versions of soluble IL-18 receptors. IL-18 is detected by Western blotting. This method provides a qualitative measure of the interaction between IL-18 and soluble IL-18 receptors.

For example, an RDI rat α-mouse IL-18 monoclonal antibody or a rat IgG2a isotype control antibody are mixed with 30 μl of (50% v/v) protein A/G agarose beads (Sigma) for 1 hour at 4° C. Beads are washed 3× with cold PBS and resuspended in 0.5 ml PBS with 1M NaCl. Mouse IL-18 (10 ng) is added and the mixtures are incubated for 1 hour at 4° C. Beads are washed and the immunoprecipitated proteins are resolved by SDS-PAGE. Western blotting is performed using a Torrey Pine rabbit α-mouse IL-18 polyclonal antibody.

(E) BIAcore. The BlAcore assay, as described in Thomassen et al. (1998) J. Ifn. Cyt. Res. 18:1077-1088), provides a quantitative measure of the interaction between IL-18 and soluble IL-18 receptors.

5. Soluble IL-18 receptor-leucine zipper heterodimers

“PCR sewing” is used to generate DNA sequences encoding chimeric mouse IL-18 receptor proteins. For example, the extracellular region of mouse IL-18R (AA1-314) is fused to the leucine zipper (LZ) region (AA161-199) of the human c-FOS protein. The extracellular region of mouse AcPL (AA20-344) is fused to the LZ region (AA277-315) of the human c-JUN protein, and inserted into pFLAG-CMV-1 (Sigma, Inc.), so that this fusion protein contains an N-terminal FLAG tag. Expression vectors encoding the IL-18R-LZf and AcPL-LZj chimeric proteins are transiently transfected into 293 cells. Cell extracts are prepared after 48 hours, and the FOS-JUN heterodimer is purified using a α-FLAG antibody column. The presence of both mouse IL-18R and AcPL in the LZ heterodimer are confirmed by Western blotting.

Human 293 cells are transiently transfected with FLAG-AcPL-LZjun, IL-18R-LZfos, or both using Effectene (Qiagen). Cell extracts are prepared and protein samples (5 μl) are resolved by SDS-PAGE. FOS-TUN heterodimers are immunoprecipitated from the double transfection extract by an α-FLAG antibody (Sigma) and eluted by a FLAG peptide (Sigma). Samples (5, μl) of the purified heterodimer are also resolved in the same gel. After Western transfer, the blots are probed with either α-FLAG (Sigma) or α-FOS (Santa Cruz) antibodies.

6. Soluble IL-18 receptor-Fc heterodimers

The extracellular regions of mouse IL-18R and AcPL, as described in example 5, are fused to the mouse immunoglobulin (subtype) Fe (starts from AA 221, hinge area between CH1 and CH2) and inserted into baculovirus expression vectors. Recombinant baculoviruses are generated and the fusion proteins are co-expressed in Sf9 insect cells. Dimeric IL-18 receptors are purified using α-Fc and α-FLAG antibody columns. The purified protein preparation consists of a population of AcPL homodimers and IL-18R/AcPL heterodimers. By infecting cells with 3×more IL-18R-expressing virus as compared to AcPL-expressing virus, the yield of heterodimers can be enhanced. The presence of soluble IL-18 receptor-Fc heterodimers is confirmed by Western blotting.

Sf21 cells are infected with IL-18R-Fc, FLAG-AcPL-Fc, or both in a molar ratio of ˜3:1. Supernatants from double infections are harvested 48 h later and are applied through a α-FLAG antibody column (Sigma). Supernatant samples, column flow-through, column wash, and eluate are resolved by SDS-PAGE. Western blotting is performed using a α-mouse Fc antibody (Sigma). A non-denatured eluate sample is also resolved by SDS-PAGE.

7. In vitro assays and in vivo models to test IL-18 receptor heterodimers

(A) In vitro. Assays to test the ability of soluble IL-18 receptor heterodimers to inhibit IL-18 in vitro include those described in examples 2, 3 and 4 above.

(B) In vivo. Models to test the ability of soluble IL-18 receptor heterodimers to inhibit IL-18 in vivo include: liver damage in P. acnes/LPS-treated nude mice (Okamura et al. (1995) Nature 378:88-90); PLP-induced, adoptive transfer experimental autoimmune encephalomyelitis in SJL mice (Leonard et al. (1995) J. Exp. Med. 181:381-386); and type II collagen-induced arthritis in DBA/1 mice (Gracie et al. (1999) J. Clin Invest. 104:1393-1401).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modification of the invention to adapt it to various usages and conditions.

We claim:

1. A soluble, heterodimeric interleukin 18 (IL-18) receptor molecule comprising two subunits, one of which comprises an extracellular domain, or a fragment thereof, of IL-18R, and the other of which comprises an extracelluar domain, or a fragment thereof, of AcPL.

2. The soluble, heterodimeric receptor of claim 1, which binds to IL-18 with higher affinity than does either IL-18R or AcPL.

3. A soluble, heterodimeric interleukin 18 (IL-18) receptor molecule comprising two subunits, each of which comprises a soluble portion of either IL-18R or AcPL appended to a peptide, wherein the two subunits are associated via said appended peptides.

4. The soluble receptor of claim 3, wherein each peptide is an immunoglobulin chain, or a fragment thereof.

5. The soluble receptor of claim 3, wherein each peptide is an immunoglobulin heavy chain, or a fragment thereof.

6. The soluble receptor of claim 3, wherein each peptide is part of a leucine zipper.

7. The soluble receptor of claim 1, wherein the subunits are associated by chemical cross-linking.

8. The soluble receptor of claim 1, wherein the subunits form a single polypeptide chain.

9. The soluble receptor of claim 1, wherein the IL-18R and AcPL are human.

10. A pharmaceutical composition comprising a soluble heterodimeric IL-18 of claim 1 and a pharmaceutically acceptable carrier.

11. A method of making a soluble heterodimeric receptor of claim 1, comprising associating the two subunits by cross-linking them chemically, by joining them with a peptide linker, or by forming a single linear polypeptide chain by recombinant methods.

12. A composition for making a soluble dimeric receptor, comprising a chimeric polynucleotide which comprises a coding sequence for a fusion protein comprising a soluble portion of IL-18R appended to a peptide, and a chimeric polynucleotide which comprises a coding sequence for a fusion protein comprising a soluble portion of AcPL appended to a peptide.

13. An expression vector comprising the chimeric polynucleotides of claim 12.

14. A host cell comprising an expression vector of claim 13.

15. A method of making a soluble heterodimeric receptor, comprising culturing a cell of claim 14 under conditions in which both of said fusion proteins are expressed, and harvesting said proteins.

16. A polynucleotide which encodes a soluble receptor of claim 8.

17. An expression vector comprising a polynucleotide of claim 16.

18. A host cell comprising an expression vector of claim 17.

19. A method of making a single chain soluble heterodimeric receptor, comprising culturing a cell of claim 18 under conditions such that single chain heterodimeric receptor is expressed, and harvesting said protein.

20. A method for inhibiting the effects of IL-18, comprising administering a soluble, heterodimeric IL-18 receptor of claim 1 to a mammal.

21. A method of treating a pathological condition associated with IL-18 expression or with excessive or inappropriate activity of cells possessing IL-18 receptors, comprising administering to a patient in need of such treatment an effective amount of a soluble, heterodimeric IL-18 receptor molecule of claim 1.

22. The method of claim 21, wherein the patient is human.

23. The method of claim 21, wherein the pathological condition is an autoimmune dysfunction or an inflammatory condition.

24. The method of claim 21, wherein the pathological condition is rheumatoid arthritis or multiple sclerosis.

25. A method for suppressing IL-18 mediated inflammation or an IL-18 mediated immune response in a mammal, comprising administering to a patient in need of such treatment an effective amount of a soluble, heterodimeric IL-18 receptor molecule of claim 1.

26. The method of claim 25, wherein the mammal is human.

27. A method of detecting an IL-18 molecule, comprising contacting a sample which may contain IL-18 molecules with a soluble, heterodimeric IL-18 receptor molecule of claim 1, which is labeled.
